(19) United States
(12) Patent Application Publication
(10) Pub. No.: US 2009/0317859 A1
(43) Pub. Date: Dec. 24, 2009

(54) CALORIMETRIC ASSESSMENT OF MICROORGANISMS AND USE THEREOF

(76) Inventors: Alma U. Daniels, Riehen (CH); Dieter Wirz, Lausen (CH); Andrej Trampuz, Basel (CH)

Correspondence Address: JOYCE VON NATZMER;PEQUIGNOT + MYERS LLC, 200 Madison Avenue, Suite 1901, New York, NY, 10016, US

(21) Appl. No.: 11/996,365

(22) PCT Filed: Jul. 24, 2006
(86) PCT No.: PCT/IB06/02009

§ 371 Date: Oct. 20, 2008

Related U.S. Application Data
(60) Provisional application No. 60/701,510, filed on Jul. 22, 2005.

(51) Int. Cl. C12Q 1/04; C12M 1/00; G06F 19/00

(52) U.S. Cl. 435/34; 435/287.1; 702/19

(57) ABSTRACT

Methods, integrated kits and systems for the rapid detection, identification and/or quantification of microorganisms in samples such as blood samples via calorimetry are disclosed.

### CROSS-REFERENCE TO RELATED APPLICATIONS

This application claims the benefit of U.S. provisional application No. 60/701,510 filed Jul. 22, 2005 which is incorporated herein in its entirety.

### FIELD OF THE INVENTION

The present invention is directed at a method, integrated kits and systems for the rapid assessment of the growth, growth rate, identity and/or quantity of microorganisms in a sample such as a blood sample.

### BACKGROUND

A common approach for determining the presence and type of microorganisms in clinical samples is based on their growth in a culture medium, followed by visual observation through the naked eye, by microscope, and/or by using biochemical analysis.

Typically, the specimen is inoculated on several agar plates and in broth media, incubated at 37° C. and examined daily for microbial growth (i.e. replication). Visible growth may require several days. Once growth is detected (at least 10,000 cells), “Gram” staining of the colony is performed to determine bacterial shape and coloration (red or blue), providing partial identification. Subsequently, an isolated colony is subjected to a complex identification process for presence or absence of distinctive enzymes. This identification process may take another 1 to 4 days to complete. If more than one microorganism grows on the plate, an isolated colony of each type must be cultured and analyzed separately.

Testing whether the microorganism is susceptible to antibiotics is generally even more complex. This process generally requires growth of the microbes on plates or in broth media. Antibiotic resistance is determined by growth of a defined bacterial cell concentration in the presence of a defined concentration of antibiotic. The process to determine antibiotic resistance requires an additional 1 to 3 days. Thus, the time from the collection of the sample to obtaining the desired information often takes 2 to 7 days.

In clinical practice, microbial infections in affected patients require prompt treatment. In severe cases, treatment within minutes may be required. Without a reliable diagnostic tool, the clinical practitioner is often left with making a “best guess” about the existence, identity and/or quantity of microorganisms. Further, the clinical practitioner will not have any information about the antimicrobial susceptibility or antibiotic resistant of the microorganism. Information about antimicrobial susceptibility is often crucial to effective treatment.

Over the last decade, alternative diagnostic approaches have been developed in an attempt to remedy these clinical problems. However, none of the alternative diagnostic approaches have routinely shortened the time for diagnosing infections. Molecular methods, such as polymerase chain reaction (PCR), have been adapted to identify microbial DNA or RNA. These methods are faster and more sensitive than plate or broth culture. However, DNA or RNA methods do not assess whether microorganisms are living or dead. Also, subsequent gene sequencing and analysis often require an additional 1-3 days. Molecular methods generally are not able to provide any information about antimicrobial susceptibility which is needed for treatment. Other molecular methods, such as DNA microarrays, enable simultaneous identification of hundreds or even thousands of different microbial genes. However, most bacterial genes encoding antimicrobial resistance are unknown, and new mutations occur constantly. Therefore, molecular methods often cannot replace, but only supplement, current culture methods.

Some more recent approaches to detecting microorganisms in a sample have been described, e.g., in U.S. Pat. No. 5,843,699 to Strenkoski, in International Patent Publications WO 2004/090089 A1 and WO02/48398 A2 and in Japanese Patent Publication No. 2003125797A.

All references mentioned in the appended bibliography are incorporated herein by reference in their entirety. Additional references, including patent references, mentioned within the body of the present text, in particular, to provide additional details respecting the practice of the invention are also incorporated herein by reference.

Studying microorganism metabolism and growth via calorimetry has been described. A partial bibliography is appended. One of these references, namely Charlebois et al. (2002), used this approach to study the metabolic response of cultured macrophages to metal and plastic particles. In fact, as noted by James (1987), the first literature reports about the use of calorimetry dates from 1912 and 1929.

There remains a need for a highly reproducible, simple, safe and/or fast method, tool and system for assessing the presence, identifying and/or quantifying microorganisms. In particular, there remains a need for a tool that allows the use of an optimized micro-/nano-calorimetry instrument in combination with a media kit-based set of optimized thermal data generation and analysis procedures. Advantageously such a tool should allow for rapid clinical and commercial detection, identification and/or quantification of microorganisms and rapidly assess the efficacy of potential tools (e.g., antimicrobials) for combating those microorganisms.

### BRIEF DESCRIPTION OF THE FIGURES

FIG. 1 shows an embodiment of a calorimeter that can be used in connection with the present application.

FIG. 2 shows heat flow rate signals from two different ampoules containing Staphylococcus aureus in 3 ml trypti-case soy broth (TSB) at a concentration ˜10⁵ cfu/ml (left) and heat flow rate signals from two different ampoules containing Staphylococcus epidermidis in TSB (˜10⁵ cfu/ml). The “flat line” data is from an ampoule containing sterile TSB as a negative control.

FIG. 3 depict heat flow patterns of Staphylococcus epidermis in 3 ml soy broth at 37 C.

FIG. 4 depicts heat flow rate signals from nanocalorimeters (Ch 1,2,3) and microcalormeters (Ch 4-1,4-2,4-3,4-4,4-5,4-6) are signals from microcalorimeters. Ch 1 Propionibacterium acnes (ATCC 11827), Ch 2 Mycobacterium fortuitum (clinical isolate), Ch 3 Candida albicans (ATCC 14053), Ch 4-1 Staphylococcus epidermidis (ATCC 12228), Ch 4-2 Staphylococcus epidermidis (clinical isolate TT 461), Ch 4-3 Escherichia coli (ATCC 25922), Ch 4-4 Pseudomonas aeruginosa (ATCC 27853), Ch 4-5 Staphylococcus aureus-MRSA (ATCC 43300), Ch 4-6 Staphylococcus aureus-MSSA (ATCC 29213).

FIG. 5 depicts an expanded scale display of data from Mycobacterium fortuitum (Ch 2) as shown in FIG. 4.

FIG. 6 provides an example of how it is possible to use our invention to determine the effects of antimicrobials (e.g. antibiotics) on the growth of microorganisms in sealed ampoules containing culture medium plus various concentrations of antibiotic. In the example shown, our invention is able to distinguish between susceptible (MSSA) and resistant (MRSA) strains of Staphylococcus aureus. Specifically: at low antibiotic concentrations (or with no antibiotic—not shown) calorimetric assessment shows that MSSA actually enters exponential growth about an hour sooner than MRSA. Conversely and definitively, at a higher concentration of antibiotic, growth of MSSA is completely suppressed, and growth of MRSA, although delayed, is only slightly suppressed. Kit software can analyze the heat signals, find these differences and report them.

### BRIEF SUMMARY OF THE INVENTION

The invention is in one embodiment directed at a method for assessing microbial growth in a sample comprising

(a) providing at least one medium selectively promoting or suppressing growth of at least one microorganism or at least one type of microorganisms,

(b) combining an aliquot of said sample with said at least one medium to produce a sample mixture,

(c) collecting heat flow signals from said sample mixture over time to produce a heat flow signal curve,

(d) determining from said heat flow signal curve whether an indicator function indicative of growth, growth rate, identity or quantity of said at least one microorganism or at least one type of microorganism exists, and if so,

(e) processing said indicator function to determine growth, growth rate identity and/or quantity of said of at least one microorganism or at least one type of microorganism.

The invention is also directed at a kit for assessing microbial growth in a sample comprising:

(a) at least one media selectively promoting or suppressing growth of at least one microorganism or at least one type of microorganisms,

(b) at least one receptacle for combining said at least one media with an aliquot of said sample to produce a sample mixture,

(c) instructions to collect heat flow signals from said sample mixture over time to produce a heat flow signal curve,

(d) a software component comprising

(i) a determining function that determines whether said heat flow signal curve displays an indicator function indicative of growth, growth rate, identity or quantity of said at least one microorganism or at least one type of microorganism, and

(ii) a processing function that processes any indicator function of (i) to determine growth, growth rate, identity and/or quantity of said at least one microorganism or at least one type of microorganism.

In another embodiment, the invention is directed at a system for assessing microbial growth in a sample comprising:

(a) at least one media selectively promoting or suppressing growth of at least one microorganism or at least one type of microorganisms,

(b) at least one receptacle for combining said at least one media with an aliquot of said sample to produce a sample mixture,

(c) a calorimeter that collect heat flow signals from said sample mixture over time to produce a heat flow signal curve,

(d) a software component comprising

(i) a determining function that determines whether said heat flow signal curve displays an indicator function indicative of growth, growth rate, identity or quantity of said at least one microorganism or at least one type of microorganism, and

(ii) a processing function that processes any indicator function of (i) to determine growth, growth rate, identity and/or quantity of said at least one microorganism or at least one type of microorganism.

### DETAILED DESCRIPTION OF THE INVENTION

The present invention draws from knowledge of (a) calorimetric instrument principles and measurements; (b) clinical and related commercial needs for rapid and accurate identification of microorganisms or exclusion of their presence; (c) behavior of microorganisms in different, controllable environments; and/or (d) responses of such organisms to methods or agents intended to inhibit growth and/or render them ineffective or inactive.

The invention makes use of one or more of a number of principles, including:
(a) microorganisms produce heat due to metabolic and replication activity;
(b) the heat flow signal (or heat flow rate signal—in micro—or nano-joules/sec; i.e. microwatts or nanowatts) produced in a sample that initially only contained a few microorganisms (MOs) will, in appropriate growth media, become quickly, accurately, and continuously detectable by micro- or nano-calorimetry, and;
(c) comparison of the nature of variation in the heat flow signal with time in various media over minutes and hours provides information about the presence, identity and/or amount of microorganisms, and in certain cases, their response to antimicrobial agents and/or sterilization/disinfection procedures;

A number of elements are used in certain embodiments of the invention, namely:

(A) The Instrument

A calorimetric instrument (or “calorimeter”) comprising 1, 2 or advantageously an array of separate calorimetric chambers for example 6, 12, 18, 24, 36, 48, 60, 72, 84, 96 or more can be used in the present invention. The chambers may or may not operate under the same temperature control regime.

Each of such chambers is in a preferred embodiment capable of, preferably at any time, and independent of the other chambers:
(a) receiving a receptacle (referred to herein just as “ampoule”) such as an ampoule for a sample mixture comprising a sample such as a body fluid or tissue suspected to contain or containing microorganisms and a growth promoting or suppressing medium;
(b) bringing the ampoules and their contents to a specified temperature within, e.g., less than about 5 minutes, about 10 minutes, about 15 minutes, about 20 minutes, about 30 minutes, about 40 minutes, about 45 minutes, less than one hour or about one hour after insertion into the calorimetric chamber;
(c) determining thermal activity of the sample mixture in the nanowatt (nW) and/or microwatt (μW) range, preferably continuously, as a function of time. Determining thermal activity can, for example, be accomplished by a direct measurement of heat flow or the electrical energy exchange required to maintain the specified temperature.

An example of a compatible calorimeter is an item 3104-2 TAM III Thermostat 230V 48 Channel (Water bath version) equipped with four of Item 3209-3 (12-Channel Minicalorimeter Set for 4 ml Glass Ampoules) (Thermometric AB), providing a total of 48 independent microcalorimeter chambers for receiving and assessing heat flow from the ampoules. These microcalorimeters have a detection limit and sensitivity of ˜0.3 microwatts (μW). In a preferred embodiment, all calorimeter chambers are maintained at the same temperature. TAM III thus essentially comprises 48 separate calorimeters all located in one thermostat which maintains them all at the same temperature with high precision. For extremely slow-growing MOs, it may be useful to use more sensitive calorimeter units in the thermostat. For example a TAM III Thermostat can be fitted with four Thermometric AB Item 3201 4 ml Nanocalorimeter units each having a sensitivity and detection limit of ˜0.03 μW. Other compatible calorimeters are possible. However, in a preferred embodiment multi-chamber instruments are used that have similar or better detection limits and stability than the one described herein. Accordingly, microcalorimeters with detection limits, such as about 0.2 microwatts and about 0.1 microwatts or less and nanocalorimeters with detection limits, such as about 0.02 microwatts and about 0.01 microwatts or less are also within the scope of the present invention.

In many cases, all that is needed to collect the data of interest is a simple sealed or sealable glass ampoule(s) in which the sample mixture is placed. For some procedures (e.g. the assessment of efficacy of antimicrobial agents or sterilization/disinfection procedures) it may be useful if quantitative, time-controlled injection into the ampoule (or flow through the ampoule) of either microorganisms or antimicrobial agents is possible after the ampoule is placed in a calorimeter chamber. However, many of the calorimetric methods previously described use a “flow-stop” technique. A culture medium containing bacteria is first poured into an electrically-driven pump and pumped through capillary tubing into a calorimetry chamber. Pumping is stopped when the chamber is filled, and the bacterial heat flow signal is recorded. This pumping technique adds substantial expense and complication to the process of obtaining calorimetric data. In a clinical setting, multi-chamber calorimeter are often used for testing multiple sample(s). Employing “a flow-stop” technique would require installing a pump for each of these chambers. In addition, the “flow-stop” technique is associated with safety hazards, since its use of pumps and tubing increases the possibility that microorganism will escape from the system.

Often it will suffice that the instrument as a whole be capable of maintaining all the chambers at the same constant temperature (e.g. 37° C.). In some cases it may benefit analysis if the instrument is capable of programmed change in temperature of the chambers as a group over time. The instrument as a whole is, in a preferred embodiment, also capable of displaying and/or storing the thermal data (typically on computer screens and/or as computer files) for analysis.

As stated above, an instrument that can be used in the context of the present invention is, for example, “TAM III” by Thermometric AB (Jarfällä, Sweden). However, instruments optimized for the purpose of this invention are envisioned and within the scope of the present invention. In certain advantageous embodiments, the instrument can bring an ampoule to the temperature of interest in a very short time such as in about 1, 2, 3, 4, 5, 6, 7, 8, 9, 10, 11, 12, 13, 14 or 15 minutes or within certain time ranges such as about 1 to 5 minutes, about 6 to 10 minutes, about 11 to 15 minutes or about 16 to 20 minutes. In certain embodiments, the calorimeter allows agitation of sample mixtures.

(B) Thermal Data Generation

A second element that is used in embodiments of the present invention is a set of thermal data generation procedures that, in a preferred embodiment, allow one to accomplish earliest possible detection, identification and/or quantification of microbial growth from the data collected via the calorimeter. Data generation procedures advantageously include the selection and use of various media promoting or suppressing growth and combining such media which the sample that might contain a microorganisms to create a sample mixture. The absence of any heat flow signal (above the baseline for an appropriate media) indicates that no microorganism that produces heat is present in a detectable quantity. If the minimum number of microrganisms to foster growth in a culture medium is initially present and growth (replication) occurs at normal rates, there are soon sufficient microorganisms for detection. Also, a given microorganism responds differently (in metabolic and replicative behavior over time) to different media promoting or suppressing growth. Therefore, for a given microorganism, there may be a distinct, medium dependent spectrum of heat flow signals and/or heat flow signal curves and/or aspects of the signals or curves. This spectrum or aspects of them may be quite different among microorganisms, which provides a basis for their identification, which might, e.g., be up to the species level and/or classification, or which might be, e.g., up to the group level. In certain embodiments the thermal data generation procedure is facilitated by diagnostic media kits designed to identify types of microorganisms in a sample and/or the effectiveness of, e.g., antimicrobial agents. Such kits comprise, in a preferred embodiment, various combinations of growth promoting and/or suppressing media (e.g., selective and/or enrichment media) for the enhancement or suppression of growth or metabolic activity of specific microorganisms. Special kits facilitate identification of multi-drug resistant microorganisms (e.g. methicillin-resistant Staphylococcus aureus, vancomycin-resistant enterococci), fungi or mycobacteria. Another type of kit comprises media which facilitate detection of mixed infections which are often missed by cultures due to lower sensitivity and low selectivity. Still other kits also comprise antimicrobial or antitumor agents to facilitate the identification of therapeutic treatment modalities.

(C) Thermal Data Analysis

A third element may be a set of thermal data analysis procedures. Various aspects of the change in heat flow signal over time can be analyzed to detect, identify and/or quantify microorganisms. For example, as mentioned above, absence of any heat flow signal (above the baseline for, e.g., an appropriate media) indicates that, e.g., no live microorganism is present in a detectable quantity. In the first few minutes and hours, the rate of increase in the amplitude of the heat flow signal (slope of the heat flow signal vs. time “curve”) is, in certain embodiments, proportional to the rate of increase in the number of, e.g., organisms (i.e., colony-forming units, cfu) present. This may be independent of the number of, e.g., microorganisms initially present. However, the time at which the slope of the heat flow signal curve begins to rise rapidly (lag time) is inversely proportional to the number of organisms initially present. The early rate of increase (slope of the heat flow signal curve) in a given medium is generally different for different organisms/cell types. The rate of increase generally does not remain linear, the rate may also temporarily decrease and the nature of non-linearity (i.e., shape of the “curve”) may be different for different organisms/cell types. In a sealed ampoule containing media and an organism, the heat flow rate may reach a peak and decline, and the maximum may also be characteristic of a given organism in a given medium. The maximum is relatively independent of the amount of organism initially present as will also be illustrated in the Figures that are discussed below.

In certain embodiments of the invention, depending on the type and amount of organism initially present, the invention offers the possibility to detect, identify and/or quantify both, e.g., microorganisms and their responses to, e.g., antimicrobial treatment within times ranging from one hour to less than one day. However, detection time and identification times of 15 minutes or less, 30 minutes or less, 45 minutes or less, 1.5 hours or less, 2 hours or less, 3, hours or less, 4 hours or less, 5 hours or less, 6 hours or less, 7 hours or less, 8 hours or less, 9 hours or less, 10 hours or less, 12 hours or less, 18 hours or less are also within the scope of the present invention. Those times might include either only detection, only identification or both detection and identification. Quantification (determination of lag time (initial concentration of microorganisms) or area under the curve (final number of microorganisms)) in similar or the same time frames are also within the scope of the present invention. This rapidity has enormous implications not only for reducing illness, improving outcome and saving lives but also for reducing the cost and length of hospital stays. By comparison, many microbiological culture methods require 2-5 days to detect and identify microorganisms. Also, quantification of the microbial burden in cultures is generally performed only semi-quantitatively or not at all, as this procedure is time-consuming and relatively inaccurate. Rapid identification of antimicrobial agents is also advantageous in many settings. A main characteristic of, e.g., microorganisms is rapid replication with cell division (replication) times below 1 hour when incubated in appropriate growth media (containing appropriate nutrients) and appropriate environment (e.g. temperature, oxygen tension). In certain embodiments, the invention quickly identifies this process as a rapid increase in the magnitude of the heat flow signal. Conversely, absence of the heat flow signal (above the baseline for an appropriate media) can rapidly and with great certainty exclude an infection; assessing the presence or absence of an infection is a common dilemma that confronts clinicians in clinical settings.

In certain embodiments, the present invention comprises (a) inoculating a media with a sample, wherein the media may be located in a sealed ampoule that is part of a kit (for example, the sample may be injected into the ampoule with needle that punctures a rubber cover of the ampoule), (b) placing the ampoule in a calorimeter and (c) acquiring thermal data for analysis, wherein the last step may be fully automated. Thus, these embodiments are characterized by their procedural simplicity. By comparison, many microbiological methods that are currently employed usually require specimen processing by a trained microbiology technician in a biosafety cabinet level type II and interpretation by a microbiologist.

In certain embodiments that are characterized by their analytical specificity, the invention determines detailed kinetic characteristics of, e.g., microorganisms (e.g. replication speed, rate of toxin production, metabolic activity of microorganisms in chronic infection). If, e.g., an antimicrobial agent is introduced and the increase in the heat flow signal declines, it is known immediately that an effective agent has been found. In addition, interactions between two or more, e.g., antimicrobial agents on, e.g., a specific microorganism can be extensively studied (e.g. additive, synergistic or antagonistic action), as well as many other important pharmacodynamic parameters (e.g. post-antibiotic effect, inoculum effect). Such information can improve current under-standing of the microbiology and pathogenesis of infections, thus have a great impact on treatment approaches. By comparison, methods for studying killing characteristics of antimicrobial agents that are currently widely employed, i.e., constructing time-kill curves, involve quantitative cultures performed at several (discrete) time points. Such methods are less accurate, are time-consuming and do not allow continuous measurements.

In certain embodiments that are characterized by their safety, the, e.g., microorganism is in a sealed ampoule of the diagnostic media kit during the entire time of data acquisition. Therefore, generally no or few safety precautions (other than, for example, avoiding dropping an ampoule and breaking it) are necessary during the analysis. By comparison, many microbiological methods require processing of clinical specimens in a biosafety cabinet level type II, direct examination of cultures on a regular basis (usually daily), sub-culturing microorganisms on new or additional growth media etc. In addition, using sealed ampoules for heat flow signal detection minimizes the risk of sample contamination (with subsequent false-positive results), which is of concern during laboratory procedures involving extensive and direct processing.

In certain embodiments of the present invention the sample remains subsequent to its use according to the invention available, that is, the calorimetric assessment does not consume or alter the sample, and it is thus available afterwards for analysis by other means (e.g., molecular detection of nucleic acid) or use for other purposes (e.g., culture or immunological techniques).

In certain embodiments, the invention's rapidity, safety, simplicity and resultant efficiency, improves patient care as it allows infections to be treated days sooner and more effectively. Earlier and targeted antimicrobial treatment substantially reduces morbidity and mortality. For example, blood products, donor tissues and organ biopsies may be tested for presence of, e.g., microorganism before infusion or transplantation. Transplant-associated infections are and have always been a pressing problem. In orthopaedic surgery, it may be possible to determine during surgery, whether tissue around the implant has become infected, or by calorimetric analysis of sonicate fluid from removed implants whether microorgansims have become attached to the implant, and if so, which antibiotic will be effective. Furthermore, persons returning from tropical countries may be screened for malaria, caused by blood protozoa of the genus Plasmodium, replicating in erythrocytes every 48 to 72 hours. Because of delayed diagnosis, malaria is still the most common cause of death from infections in travellers returning from tropical countries. Exclusion of malaria may also shorten the current 6-month observation period after return.

In other embodiments, the invention is used for screening for pulmonary tuberculosis in refugees by testing their sputum for presence of Mycobacterium tuberculosis. Currently border control procedure is often a chest x-ray: an expensive and relatively inaccurate procedure.

In certain embodiments, the invention reduces the risk of spread of multi-resistant pathogens. The embodiments allow patients carrying resistant microorganisms, such as methicillin-resistant Staphylococcus aureus (MRSA), to be screened and identified as such at hospital admission. This allows isolating and decolonizing carriers immediately, thereby substantially reducing the risk of transmission of resistant bacteria to healthcare workers and other patients. Such an early and accurate diagnostic tool for multi-resistant pathogens greatly improves current epidemiological and infection control practices and promises to make eradication of multi-resistant pathogens possible.

Cost reduction is another feature of certain embodiments of the present invention. The potential saving in labor and thus medical costs are large. For example, patients must sometimes be kept in expensive isolation for days until it is determined that they are not infected with antibiotic resistant bacteria. Also, the hardware components to practice the present invention are in their basic configuration already in existence. Thus, the basic calorimetric instrumentation technology already exists as well as the hardware needed in connection with those instruments to practice the present invention, such as, e.g., glass ampoules that can be readily set up to contain specific sets of ordinary growth media and antibiotics.

In certain embodiments, the invention is directed at early detection of rapidly replicating microorganisms in primarily sterile body fluids, such as blood (bacterial or fungal sepsis, malaria) or cerebrospinal fluid (meningitis). These infections are usually emergencies with high mortality rates and therefore require rapid identification and initiation of empirical treatment. Even in the absence of microbial identification, the invention enables confirmation of the treatment efficacy by repeated measurement of the heat flow signal in additional clinical samples, collected after beginning antimicrobial treatment.

In other embodiments, the invention is directed at early detection of slowly or non-replicating microorganisms, which often require weeks or months to be detected with other methods. Such microorganisms include mycobacteria, brucellae, actinomycetes, or unidentified microorganisms (suspected as causes for diseases currently of unknown etiology). Other difficult-to-detect microorganisms include “nutritionally variant streptococci” (Abiotrophia defectiva, Granulicatella adiacens) and small colony variants of staphylococci, which may cause chronic and recurrent infections. Rapidly observing a beginning heat flow signal/time curve characteristic in certain respects of a (e.g., initial slope, subsequent curve shape or peak, overall shape of the curve to determine area under the curve) is likely to allow rapid identification of such microorganisms, in particular, but not necessarily, when coupled with supplemental clinical, laboratory and radiological assessments.

In certain embodiments the invention allows for accurate exclusion of the presence of microorganisms. In many situations such information is crucial, e.g. donor-based transfusion medicine (e.g., whole blood, plasma, concentrates of erythrocytes or platelets), allogenic or xenogenic transplantation (organs or tissues), surgery involving artificial devices (medical or surgical), sterilization and disinfection control (e.g. surgical instruments, endoscopes) etc.

While the above description focused on a clinical setting, the present invention has equal applicability for the food, beverage, pharmaceutical, medical device industries or any other industry have a need to detect, identify and/or quantify microorganisms in their procedures and products.

There are many variables that deserve attention in different embodiments of the present invention. These variables will be described herein in reference to kits of the present invention and a calorimeter used in conjunction with those kits. However, the following descriptions are in general equally applicable to embodiments of the methods and systems of the present invention.

In certain embodiments, the kit comprises instructions which describes how the kit components are used in conjunction with a calorimeter and how the data acquired via the calorimeter is further analyzed and processed.

Handling of the Sample and Equipment:

In certain embodiments, a compatible calorimeter instrument should be first stabilized at a specified temperature, for example 37° C. The calorimeter chambers are hereby, in certain embodiments, calibrated to produce accurate measurements of heat flow signals at the set temperature. Also, in certain embodiments any difference between the heat flow signal from an internal, thermally-inert reference and each of the instrument's empty chambers is eliminated; i.e., reduced to zero microwatts (μW).

To ensure that a kit and the compatible calorimeter are generally functioning properly, it is advisable in certain embodiments of the invention to prepare specimens deliberately spiked with known types and amounts of one or more MOs, provided by the kit user. One or more of the media may be employed as desired by the user.

The amount of time from insertion of the specimen into an ampoule until insertion of the ampoule into a calorimeter chamber is in certain embodiments of the present invention determined and recorded, and kept as constant as possible. This “insertion-insertion time” can be about 5, about 10, about 15, about 20, about 25 or about 30 minutes. In certain embodiments it is more, in others less.

The ampoule may be inserted into a calorimeter chamber until it reaches the instrument-specified equilibration position and left there for a specified equilibration time. This time allows the ampoule to closely approach the set temperature of the calorimeter's measurement position, such as, but not limited to 37° C. The equilibration time may be about 1, about 2, about 3, about 4, about 5, about 10, about 15, about 20, about 25, about 30 minutes. Depending on the instrument used, higher or lower equilibration times are also within the scope of the present invention.

The ampoule may then be lowered to the measurement position in the calorimeter chamber. Even after the period in the equilibration position, placement of the ampoule in the measurement position often produces thermal disturbances and an extraneous heat flow signal unrelated to the thermal activity of any microorganism in the sample mixture.

Therefore, a stabilization time which may be specified in the kit instructions may in certain embodiments of the invention be set until such extraneous heat flow signal(s) subside, and data can be acquired. The stabilization time may be about 5, about 10, about 15, about 20, about 25 or about 30 minutes. In certain embodiments higher or lower stabilization times are desirable. In certain embodiments of the invention methods to reduce this time are employed. This includes “subtraction” during the stabilization time period of the mean of signals of a group of simultaneously inserted negative controls or just signals from one simultaneously inserted negative control. Such a substraction can be performed continuously and/or as soon as the required data is available. These controls also have the effect that their calorimeter chambers are subject to essentially the same thermal disturbances and/or temporarily produce the same type of extraneous heat flow signals as those of the sample mixture.

Data Acquisition:

After the system is stabilized, data, i.e. heat flow signals, from each ampoule (heat flow rate (μW) from the specimen relative to its internal reference) as a function of time is captured and generally placed in computer-stored data files. For this process, in many embodiments the general software supplied with a compatible calorimeter is used. In a preferred embodiment, the data recorded will include sample identification information for each ampoule. Heat flow data may be recorded and/or stored from each ampoule at different rates such as 1 value each second, 1 value every 5 seconds, 1 value every 10 seconds etc. Substantially lower rates, including, but not limited to, 1 value every 30, 40, 50, 60, 70, 80, 90, 100, 110 or 120 seconds are possible. In fact, certain embodiments of the invention only require recording of heat flow values at intervals of several minutes such as, but not limited to every three, four, five, six, seven or eight minutes.

Software Component(s):

In one embodiment, the data acquired and stored in, e.g., computer files by, e.g., a compatible calorimeter's general software, may be accessed by the kit's software for analysis. The kit software can in certain embodiments reside on the same computer as the calorimeter's own software or on a separate computer. Also components of the calorimeter software can be integrated into the kit software and vise versa. The kit software is in certain embodiments “aware” of the information specified above, in particular the information specified under “Handling of sample and software” and is supplied with any resultant values (e.g., insertion-insertion time, equilibration time, stabilization time). The software may also include a key which interprets the identification information provided in the data files created by the calorimeter's general software. Where time is of the essence, the transfer of data from calorimeter software to kit software takes place in real time, that is, as the data are acquired. In a growth kit such as the one described in Example 1, the purpose of the kit is to assess, preferably in the minimum time possible, whether microorganism growth is occurring in the sample and, preferably to immediately report this finding to the user. The kit's software can accomplish this task in many ways, some of which are described in the examples.

The speed with which the desired information is outputted, in particular, information about microbial growth, depends, among others, on the detection limit of the calorimeter and on the type and initial concentration of the microorganisms (or MOs) present in the sample. In general, each living microorganism produces ˜2×10⁻¹² W (2 picowatts) of metabolic heat during replication. However, it should be noted that the actual amount of metabolic heat varies and depends on the specific type of MO, the activity in which it is engaged, e.g., replication, and its environment. Thus, the metabolic heat per cell may be much higher at some stages of replication, so that detection may well occur when much smaller numbers of cells are present. Also, there can be other living cells in blood specimens, but replication (if any) is ˜10-100 times slower than most microorganisms.

In one preferred embodiment of the invention, heat flow signal values are examined continuously, that is, e.g. 5 to 50 values or 5 to 100 values, including 10, 20, 30, 40, 50, 60, 70, 80, 90 values, of the most recently acquired heat flow signal values that have been plotted against time to create a “heat flow signal curve” are examined for indicator functions including growth, growth rate, identity and/or quantity.

Definitions

An example of a “receptacle” according to the present invention is a glass bottle that can receive 1 ml, 2 ml, 3, ml or 4 ml or more sample mixture. However, smaller sized receptacles are also within the scope of the present invention, such as receptacles that are designed to receive 0.5 ml, 0.1 ml, 0.05 ml or 0.01 ml or less are within the scope of the present invention. The receptacle can be made of many materials, including glass and polymers. In most cases a basic criterion for the receptacle material is that, in particular when it is sealed and includes a sample mixture or control and is placed in a calorimeter, it does not produce any heat flow signal (e.g., due to chemical or physical changes in the receptacle, or leakage of fluid or vapor) that is an appreciable fraction of the heat flow signals to be measured—i.e. one in the approximate range of ˜0.5-500 μW in the context of the present invention.

Generally one receptacle is received by one chamber of the calorimeter. Preferably, the receptacle has an leak-proof or essentially leak-proof sealing system, comprising, e.g., a silicone elastomer septum affixed by crimping a metal collar over it and around the neck of the receptacle, using, e.g., a special crimping tool. One type of bottle that can be used in the context of the present invention and which also includes sealing components is available as Item 2505-41 from Thermometric AB, Järfälla, Sweden as well as from other sources. The crimping tool is also available, as item 2277-306, from Thermometric AB, Järfälla, Sweden. However, many other types of receptacles are within the scope of the present invention. The receptacles may be pre-loaded with medium and be sealed. Samples may be added, e.g. by piercing the receptacle's septum with a needle. Negative control receptacles can also come pre-loaded with medium plus sterile PBS. A lifting mechanism such as a lifting eyelet or metal hook may be fixed to a sealing system for inserting the receptacle into the calorimeter and later removing it. One example is a hook in the form of a zinc-plated steel ⅙×3 closed ring with a tapered, threaded stem. A receptacle insertion/removal fixture may be incorporated directly into the receptacle design. Alternatively, a receptacle insertion/removal function may be provided by a mechanism in a compatible calorimeter. However, in general, receptacle design is largely dependent on the calorimeter with which it is used and receptacles that can be used with very small “calorimeters” are within the scope of the present invention so that multi-titer plate like receptacles are within the scope of the present invention.

A “medium” according to the present invention is any medium that promotes or suppresses growth of a microorganism (MO). Promoting hereby includes simple maintenance of growth. A medium that is said to promote growth typically comprises one or more culture media that provide nutrients for an MO. A medium that is said to suppress growth typically includes one or more growth suppressing substances such as antimicrobial substance or, e.g., substances that increase or decrease pH or bacteriophages. However, growth may also be suppressed by environmental factors such as, but not limited to, temperatures that are below or above growth maintaining temperatures or aerobic/anaerobic conditions. Substances suppressing growth are also referred to herein as “suppressor agents” and conditions suppressing growth are also referred to herein as “suppressor conditions.” The equivalent nomenclature applies to substances and conditions that promote growth, which are referred to herein as “promoter agents” and “promoter conditions,” respectively. The medium can, in particular be a culture medium such as, but not limited to, trypticase soy broth (TSB), thioglycollate broth (TGB), brain heat infusion (BHI), Barbour-Stoenner-Kelly II medium (BSK II), Middlebrook 7H9 broth, Brucella broth, University of Vermont modified Listeria enrichment broth or buffered charcoal yeast extract medium (BCYE). A medium according to the present invention may contain additives, such as antimicrobial substances including, but not limited to, benzylpenicillin, oxacillin or nafcillin, ampicillin or amoxicillin, cefazolin, cefuroxim, ceftazidim, cefepim, imipenem, meropenem, clarithromycin, doxycyclin, clindamycin,tobramycin, amikacin, netilmicin, cotrimoxazol, nitrofurantoin, norfloxacin, ciprofloxacin, levofloxacin, moxifloxacin, vancomycin, teicoplanin, fusidinic acid, rifampicin, linezolid, glycopeptides or aminoglycosides. In certain embodiments the concentration of these antimicrobial substances is higher than the minimum inhibitory concentration (MIC) for MOs of specific groups (e.g. glycopeptides suppress growth of the majority of Gram-positive MOs and aminoglycosides suppress growth of the majority of Gram-negative MOs, azoles suppress growth of the majority of fungi). Other additives are non-antimicrobial substances such as hypertonic saline, or substances causing extreme pH. These substances may in certain embodiments be employed to select specific MOs growing in their presence, but not others. The medium may also contain substances that do not substanially affect the heat generation properties of the medium or the respective control and/or sample mixture such as sterile phosphate buffered saline (PBS), which is often added to mediums that are part of “controls.” Other potential additives include agents for maintaining the pH of the medium (salt and tris buffers), O₂-reducing agents (L-cystein), and specific nutrients (iron, factor X, factor V).

A “sample” according to the present invention is any kind of specimen that may contain a microorganism. This includes for example body fluids such as blood, blood products such as plasma, cerebrospinal fluid, normally sterile body fluids, such as synovial fluid, amniotic fluid, peritoneal fluid (ascites), peritoneal dialysis effluent, pericardial effusion, pleural, effusion, bone marrow aspirate as well as body tissues and sonicate fluid. However, other specimens containing or potentially containing microorganisms such as food products, pharmaceutical products, waste water or drinking water are also within the scope of the present invention. Those microorganisms may be a desirable component of the sample or at least a component of the sample that is of no concern or an aberrant microorganism that is typically not desirable and generally will raise concern when found in the sample. A sample can be of varying size. The sample size will depend to a large extent on the sensitivity of the calorimetric instrument used. The higher the sensitivity of the instrument the smaller the sample size can be. The sample size also depends on the nature of the sample. For example, cerebrospinal fluid has considerably higher MO concentration than blood. Thus, smaller samples/sample mixtures may be employed. The present invention encompasses sample sizes including about 0.001 ml, about 0.005 ml, about 0.01 ml, about 0.05 ml, about 0.1 ml, about 0.25 ml, about 0.5 ml, about 1 ml, about 2 ml, about 3 ml or more or less. Samples of pediatric patients can usually be supplied in lower amounts since bacterial concentrations in children, in particular in children's blood, are typically 2× to 10× higher.

The terms “real-time determination” or “determination in real-time” of an indicator function denotes, in accordance with the present invention, data transfer which allows, determination of the presence of an indicator function as data that conveys such an indicator function, e.g., a series of heat flow signal values such as 5, 10, 15, 20, 25, 30, 35, 40, 45, 50 heat flow signal values or more is received, e.g., by a software component. “Real-time processing” allows for the immediate analysis of said indicator function.

The term “heat flow signal curve” and “heat flow signal value sequence” are used interchangeably in the context present invention. Thus, a “heat flow signal curve” will include a sequence of heat flow signals values (“heat flow signal value sequence”) that are not, but could be, connected to form a curve. A “heat flow signal curve” or “heat flow signal value sequence” results from plotting heat flow signals or more precisely a heat flow rate signals against time. Individual heat flow signals are typically measured in Watts (joule/sec) or microwatts or nanowatts. The heat flow signal curve illustrates how heat flow signals collected relate to each other over time. The information of at least parts of a heat flow signal curve according to the present invention is in many aspects of the invention used as an indicator function according to the present invention. Similarly a reference to any other curve herein, such as a growth “curve,” could equally be described as and includes a sequence of growth data points.

An “indicator function” according to the present invention is any part of a heat flow signal curve/heat flow signal curves that provide(s) information about microbial growth in the sample. This includes, for example, the lag until growth starts, an rise or fall of the growth curve, a peak in the growth curve, the overall shape of one or more curves or a combination thereof.

“Processing” an indicator function means in the context of the present invention that the heat flow signal values that make up the indicator function are processed, preferably by a computer, to assign them an information carrying value about growth, growth rate, identity and/or quantity of a microorganism or type of microorganism.

“Selectively promoting or suppressing growth” in the context of the present invention means that there is a certain degree of selectivity in the promotion or suppression of growth. In particular, the term signifies that the medium is designed to at least either promote or suppress a certain type of microorganism, for example, a gram positive, gram negative, thermopile or acidophil bacterium. In certain cases this selectivity extends for example to the bacterial genus or specie.

A “microorganism” according to the present invention includes in particular bacteria, such as, but not limited to, Streptococcus pneumoniae, Neisseria meningitidis, Mycobacterium spp., Legionella spp. and fungi such as, but not limited to, Candida spp. or Aspergillus spp. A “type of microorganism” defines a group of microorganisms that have at least one common characteristic such as gram staining or, e.g., being acidophil. This term also includes microorganisms that fall within one genus.

“Growth” according to the present invention includes any kind of quantitative or qualitative growth of such microorganisms, such as, for example, “growth” resulting from expansion of individual microorganisms as well as for the multiplication of microorganisms. A “type of microorganism” defines a group of microorganisms that have at least one common characteristic such as gram staining or, e.g., being acidophil. This term also includes microorganisms that fall within one genus.

A “control” in particular a negative control according to the present invention is a solution that consists essentially of at least one medium. While there might be other solutions such as buffer solutions in the control, the control will not include any sample unless specifically mentioned.

A “equilibration position” according to the present invention is a position in which a receptacle and its contents is brought to the temperature of the calorimeter, but at which no temperature measurements of the contents are performed. The “equilibration time” is the time required for the receptacle and its contents to reach the temperature set by the calorimeter.

A “measurement position” according to the present invention is a position in which heat flow signals from the contents of the receptacle are measured. The “stabilization time” is the time required for thermal disturbances and/or extraneous heat flow signals unrelated to the thermal activity being produced by any microorganism in the sample mixture to subside after the receptacle is brought into the measurement position.

When a difference between values of heat flow signal curves becomes “significant” (“significant difference”) will depend on the instrument used. However, generally, a difference is considered significant only if it is above the detecting limit of the instrument. Differences that are 5 times, 6 times, 7 times, 8 times, 9 times, 10 times or more beyond the detection limit of the instrument constitute appropriate “significant differences.”

The invention will now be described with reference to Figures and Examples that describe kits exemplifying certain embodiments of the invention. These Figures and Examples are for illustrative purposes only and shall not be construed to limit the present invention. The Figures and Examples will guide the person skilled in the art to make and use the present invention. They will also allow the skilled artisan to appreciate the scope of the invention that is set forth in the appended claims.

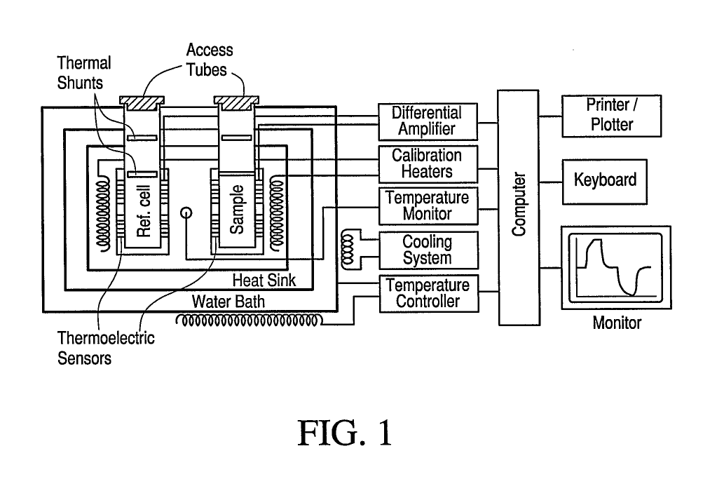

FIG. 1 shows an example of one type calorimeter and the overall set up of typical components of a calorimeter that can be used in connection with the present invention. This Figure depicts a calorimeter with two examplatory chambers, one receiving a receptacle (here designated as “access tube”) containing a sample mixture (here simply designated as “sample”) and one for a control or for, e.g., a piece of aluminum (not shown) that can serve as a reference (here designated as “Ref. cell”). The chambers are in particular equipped with thermoelectrical sensors. The calorimeter also includes a temperature monitor as well as software and, in the example shown, hardware to collect the data collected via the sensor and monitor it.

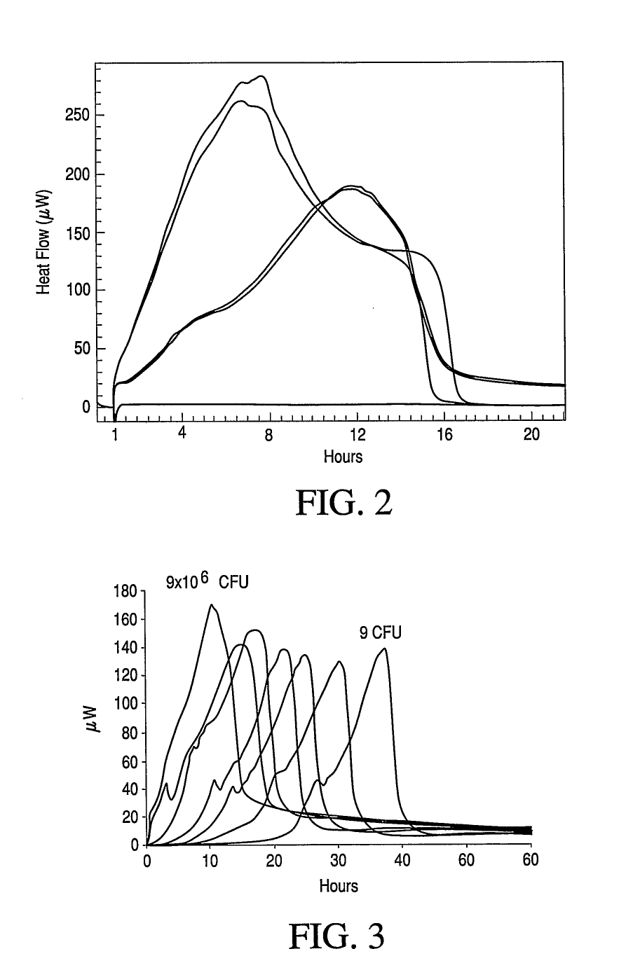

FIG. 2 shows four curves of the heat flow rate signals of two types of microorganisms. The curves with the higher and earlier occurring peaks were obtained from two sealed ampoules each containing Staphylococcus aureus in 3 ml trypticase soy broth (TSB) at a concentration ˜10⁵ cfu/ml. The curves with the lower and later occurring peals were obtained from two ampoules each containing Staphylococcus epidermidis in TSB (˜10⁵ cfu/ml). The “flat line” data around zero is from an ampoule containing sterile TSB, which serves as a negative control. Notable is the similarity of the curves for the pairs of quasi identical sample mixtures tested. This similarity demonstrates the high reproducibility of the methods of the present invention.

FIG. 3 depicts seven heat flow patterns of Stapylococcus. epidermins in 3 ml soy broth at 37° C. with initial concentrations of the bacterium varying 7 orders of magnitude (9×10⁶ CFU to the left and 9 CFU to the right with step-wise increasing concentrations in between). Notable is that the initial concentration of the bacterium has little or no effect on the slopes and peaks. The curves only differ in the lag phases, with sample mixtures having lower concentration having a longer lag phase. The slopes have been determined to be proportional to the rate of increase in the number of bacteria. The peaks mark the transition to inactive state.

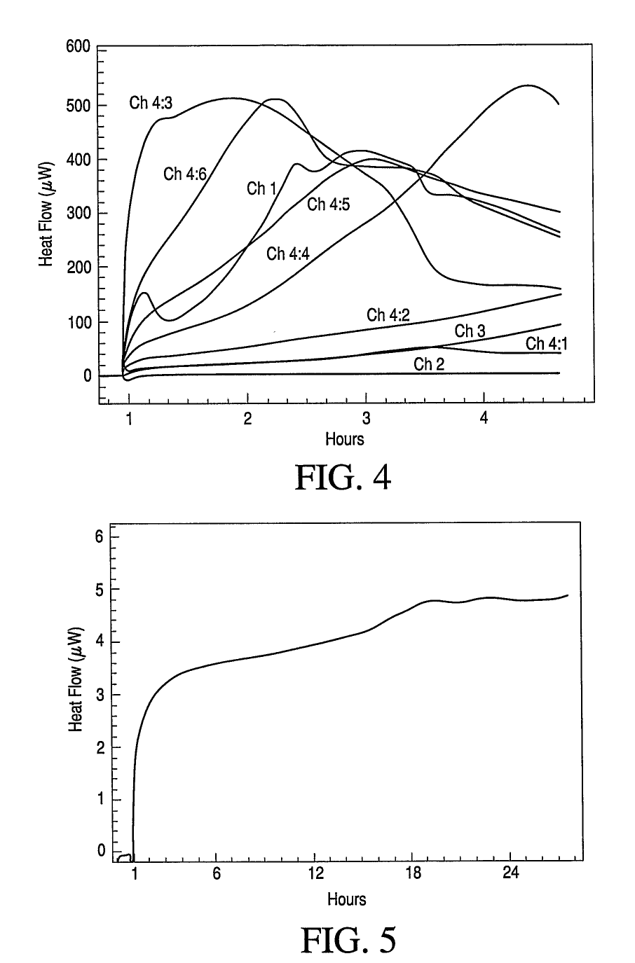

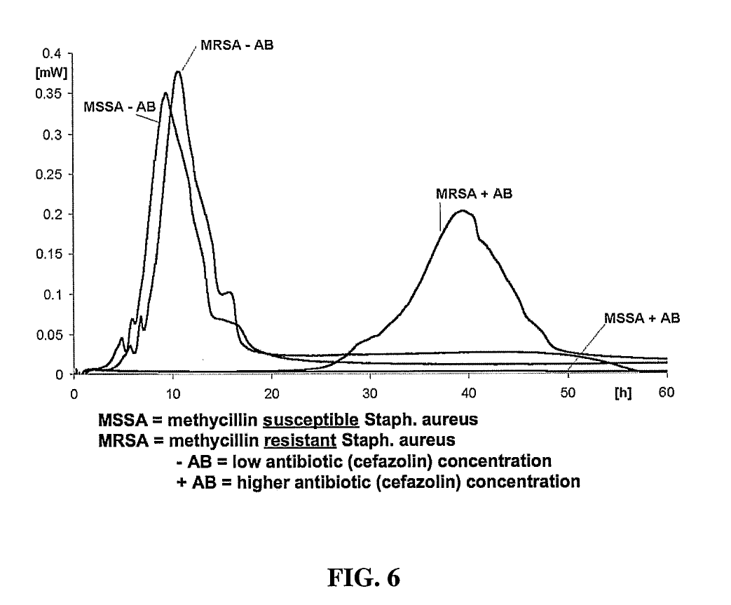

FIG. 4 depicts heat flow data obtained from a wide array of microorganisms in the same growth medium. Ch 1, 2, 3 show heat flow signals obtained with nanocalorimeters. Ch 4:1, 4:2, 4:3, 4:4, 4:5, 4:6 are heat flow signals obtained with microcalorimeters. Ch 1 signifies Propionibacterium acnes (ATCC 11827), Ch 2 Mycobacterium fortuitum (clinical isolate), Ch 3 Candida albicans (ATCC 14053), Ch 4-1 Staphylococcus epidermidis (ATCC 12228), Ch 4-2 Staphylococcus epidermidis (clinical isolate TT 461), Ch 4-3 Escherichia coli (ATCC 25922), Ch 4-4 Pseudomonas aeruginosa (ATCC 27853), Ch 4-5 Staphylococcus aureus-MRSA (ATCC 43300) and Ch 4-6 Staphylococcus aureus-MSSA (ATCC 29213). The graph shows characteristic heat flow signal curves that can be obtained for these different microorganisms.

FIG. 5 is an expanded scale display of data from Mycobacterium fortuitum (Ch 2) also depicted in FIG. 4. This graph shows that via nanocalorimetry meaningful heat flow data (e.g., presence or absence in sample) can be obtained for slower-growing organisms.

### Examples

### Example 1

### Kit for General Detection of Microorganisms in Blood Specimens

This example describes a kit that that will allow one to detect whether any of a broad array of living common microorganisms (MOs) is present and/or replicating in, e.g., blood or blood product (e.g. platelet concentrate) samples quickly and accurately. The kit detects those Gram-positive and Gram-negative aerobic and anaerobic MOs which frequently cause human bloodstream infections. In the example presented, for adults, 1 ml blood is sufficient, whereas in pediatric patients smaller volumes of blood are sufficient (0.1-0.5 ml) since bacterial concentrations in children are typically 2× to 10× higher. Simple 4 ml glass ampoules are used in this example as receptacle.

To fulfill the broad-range detection purpose, a set of general purpose, enriched, selective and specialized culture media are used in the kit's ampoules. Culture media may be pH-adjusted and supplemented with additives to facilitate or inhibit growth of specific MOs. The media for this kit may include:
(1) For aerobic and facultatively anaerobic organisms—trypticase soy broth (TSB) with 0.025% sodium polyanetholesulfonate (SPS)
(2) For microaerophillic and obligate anaerobic bacteria—thioglycollate broth (TGB) with 0.025% sodium polyanetholesulfonate (SPS), hemin and vitamin K
(3) For Mycobacterium spp.—Middlebrook 7H9 broth
(4) For fungi (e.g., Candida spp., Aspergillus spp.)—brain heart infusion (BHI) with chloramphenicol (8 μg/ml) and streptomycin (40 μg/mI)

In this example, the following procedures take place in a Class 2 biohazard facility under ambient conditions (for detection of aerobic or facultatively anaerobic MOs) or under anaerobic conditions (for detection of obligate anaerobic MOs):

(a) One or more clean sterile ampoules are first filled with 2 ml of each of the above media, which are provided as part of the kit. In other versions of this and other kits, ampoules come pre-loaded with media.

(b) After the media are in place, up to 1 ml of patient sample (undiluted or diluted with normal saline to create a 1 ml sample) is then added to each of the open ampoules.

(c) In this example, each ampoule is then sealed and is ready for micro-nano calorimetric assessment.

Instructions

The instruction component of this kit provides the following information and directions:

(a) Calorimeter:

The instrument has to be stabilized, in this example, at 37° C. In accord with the instrument's directions, the calorimeter chambers are calibrated to produce accurate measurements of heat flow at the set temperature. Also, any difference between the heat flow signal from an internal, thermally-inert reference and each of the instrument's empty chambers is eliminated; i.e., reduced to zero microwatts (μW) of relative heat flow.

(b) Handling of the Sample and Calorimeter

To ensure that a kit and the compatible calorimeter are generally functioning properly, it is advisable to prepare specimens deliberately spiked with known types and amounts of one or more MOs, provided by the kit user. One or more of the media are employed as desired by the user. In this example a 4 ml ampoule is filled with 2 ml of a given media. A known type and amount of a MO known to grow in that media is placed in PBS to create a 1 ml “sample” or “specimen” and added to the media. The ampoule is then sealed and treated in the same manner as regular sample mixtures, as described next.

In this example the insertion-insertion time is 30 minutes, the equilibration time is 15 minutes to closely approach the set temperature of the calorimeter's measurement position, in this example, 37° C.

The ampoule is then lowered to the measurement position in the calorimeter chamber. The stabilization time is in this example 30 minutes.

(c) Data Acquisition:

The instructions next dictate that after the stabilization time has elapsed, data from each ampoule (heat flow rate, μW, from the sample relative to its internal reference) as a function of time are captured and placed in computer-stored data files using the general software supplied with a compatible calorimeter. Data recorded include sample identification information for each ampoule for which data are recorded. In this example, heat flow data are recorded and stored from each ampoule at a rate of 1 value each second.

Kit-Specific Computer Software

In this example, the data acquired and stored in computer files by the compatible calorimeter's general software are accessed by the kit's software for analysis. Here, the kit software is “aware” of the procedures specified in the kit instructions and is supplied with any resultant values (e.g., insertion-insertion time, equilibration time, stabilization time) and also includes a key which interprets the identification information provided in the data files created by the calorimeter's general software. Since time is of essence in this kit, the transfer of data from calorimeter software to kit software takes place in “real time.” In this kit, the purpose is to assess in the minimum time possible whether microorganism growth is occurring in the sample and to immediately report this finding to the user. Here, the kit software accomplishes this task in two ways, described below.

In either method below, the absolute time until detection depends, among others, on the detection limit of the calorimeter and on the type and initial concentration of MO (or MOs) present in the blood sample. As described, each living microorganism produces ˜2×10⁻¹² W (2 picowatts) of metabolic heat during replication. However, it should be noted that the actual amount of metabolic heat varies and depends on the specific type of MO, the activity in which it is engaged. In this example, the calorimeter's detection limit is ˜0.3×10⁻⁹ W (0.3 μW). Therefore this calorimeter can detect the presence of approximately 150,000 replicating MOs, and also can detect any increase in the number of such cells that is greater than ˜150,000. How this translates into the microbiologic term “colony forming units” (CFUs) depends on the number of cells per CFU, which can be determined by other means. Conservatively, this example of the invention thus does not produce CFU counts but signals if there are more than ˜150,000 replicating microorganisms present. However, the metabolic heat per cell may be much higher at some stages of replication, so that detection may well occur when much smaller numbers of cells are present. As explained further below, the invention primarily detects living microorganisms by detecting their exponential growth in suitable culture media.

The primary and secondary method described in connection with this example, has, as the person skilled in the art will appreciate and optionally with modifications described herein and known to the person skilled in the art, applications far beyond this specific example.

In the Primary method, the kit software continually examines sequences, e.g. 5-50 values, of the most recently acquired heat flow signal data points to see how well the data conform to an exponential rise. This is based on the fact that after a lag phase in culture, the number of MOs begins to increase exponentially with time. As a consequence, the aggregate amount of metabolic heat detected by the calorimeter will also increase exponentially. Also, experience in developing the kits described herein has shown that no other reactions in or among the components in the kit ampoules (media, serum) produce exponentially increasing heat flow signals. In fact, slow degradation of the fixed amount of media, serum or other blood components present begin to produce heat flow signals which decline slowly and exponentially, as the amount of unreacted media still present declines. In this example, the software employs a simple strategy for detecting an exponential rise: The software repeatedly computes the logarithms of the latest sequence of heat flow signal data points. If there is an exponential rise, the change in the logarithmic values with time will be linear. Therefore the software performs a linear regression on the logarithmic data and computes R², the coefficient of determination. An R² value of 1.0 would be found if the data were perfectly linear. In this example, an increase in R² to a value above 0.9 is taken as an initial indication of an exponential rise in the heat flow signal (and thus the presence of replicating MOs), and the software reports this finding to the user. A higher R² value e.g., 0.99 or greater is considered as definitive detection of a replicating MOs, and this is also reported when it occurs. The specific value of R² considered definitive of detection depends on the culture medium and other variables of which the software is aware. Values of R² greater than 0.9 are the norm until replication is curtailed by consumption of nutrients or build up of waste products in the sealed ampoule. This method can be applied either directly to the data from a sample-containing ampoule or to differential data produced by subtracting the signal from negative controls.

A secondary method is also incorporated in the kit software. It is generally slower to signal detection but provides a confirmatory backup. In this case, the software examines the magnitude of the heat flow signal relative to signals from negative controls (i.e. ampoules containing media plus sterile serum) and provides an alert to the kit user when the signal from any sample-containing ampoule becomes “significantly higher” than the corresponding negative control. In this example, the calorimeter instrument is able to detect heat flow signal differences of ˜0.3 μW. Based on this, the detection of a difference between any specimen signal and its negative control of 10 μW (roughly 30 times the detection limit) is used in this example as a conservative estimation of “significantly higher”. Comparison of sample data from previously-described optional controls spiked with known amounts of various MOs in media known to encourage their replication also gives at least an indication of which MOs may be present and in what initial concentration.

Compared to standard microbiologic methods (e.g. culture plating and incubation at 37° C. until colonies are visible, which typically takes 16-48 hours) even the prototype kit described here generally detects replicating microorganisms in blood sample twice to ten times as fast.

For the kit examples 1 through 3 a goal is to determine positivity, i.e. to detect as quickly and as accurately as possible, whether any of a broad array of living common microorganisms (MOs) is present and/or replicating in the sample. The software analyzes heat flow and gives the kit user information for deciding whether a specimen is positive or not. In particular, the following parameters are determined directly and reported: (1) time to positively, (2) replication rate and (3) total heat.

### Example 2

### Kit for Detection of Microorganisms in Cerebrospinal Fluid

Purpose

The purpose of this kit is to detect as quickly and as accurately as possible any of the common MOs causing bacterial meningitis is present and/or replicating in cerebrospinal fluid (CSF) specimens harvested by lumbar puncture, intracisternal puncture, or puncture of a neurosurgically introduced ventricular shunt. Streptococcus pneumoniae and Neisseria meningitidis are responsible for >80% of all bacterial meningitis cases. Data supports that for these two most common MOs, the magnitudes of the maximum heat flow signals for Streptococcus pneumoniae are consistently and distinctly higher than those for Neisseria meningitidis, and that this difference is independent of initial concentration of either MO. Thus, this kit provides data for the identification of CSF MOs in addition to detection.

This kit is analogous to Example 1: Kit for General Detection of Microorganisms in Blood Specimens. The general description and specific example presented in Example 1 apply, but with the following modifications:

(a) The MOs to be detected and the kit media employed are as follows:
(1) For Streptococcus pneumoniae, Neisseria spp. and Haemophilus spp.—brain heart infusion (BHI) with Fildes enrichment additive
(2) For Borrelia burgdorferi—Barbour-Stoenner-Kelly II medium (BSK II)
(3) For Mycobacterium spp.—Middlebrook 7H9 broth
(4) For Brucella spp.—Brucella broth with sodium bisulfite as reducing agent
(5) For Listeria monocytogenes—University of Vermont modified Listeria enrichment broth

As mentioned above, the peak of the heat flow signal curve in a given medium can also be used to help identify the MO present. Specifically, in BHI, the maximum magnitude is higher for Streptococcus pneumoniae than for Neisseria meningitidis.

(b) The volume of CSF available and needed for analysis is generally smaller than for blood, because of the typically higher bacterial concentration in CSF (>10⁶ CFU/ml) than in blood (10-100 CFU/ml) and the rapid replication rate of the most frequently involved MOs (Streptococcus pneumoniae, Neisseria meningitidis and Haemophilus influenzae). This reduces potential severe adverse events to the patient (e.g. brain herniation, persistant headache), especially if multiple assessments are clinically needed (e.g. to evaluate the antimicrobial efficacy) or small children are involved (small volume of CSF available). In this example, the typical amount of CSF specimen required is 0.01-1 ml.

(c) This method provides a rapid and accurate method for detection of MOs in CSF. Direct microscopic examination of the Gram-stained CSF sediment is a fast method, but is not sensitive due to present and overlapping white blood cells (pleocythosis). Bacterial meningitis is always a medical emergency and requires a rapid diagnosis and early antimicrobial treatment to prevent morbidity and mortality.

### Example 3

### Kit for Detection of Microorganisms in Other Normally Sterile Body Fluids

Purpose

The purpose of this kit is to detect as quickly and as accurately as possible whether MOs are present and/or replicating in other normally sterile body fluids, such as synovial fluid, amniotic fluid, peritoneal fluid (ascites), peritoneal dialysis effluent, pericardial effusion, pleural effusion, bone marrow aspirate.

This kit is analogous to Example 1: Kit for General Detection of Microorganisms in Blood Specimens. The general description and specific example presented in Example 1 apply, but with the following modifications:

(a) The MOs to be detected and the kit media employed are as follows:
(1) For aerobic and facultatively anaerobic organisms—trypticase soy broth (TSB)
(2) For microaerophillic and obligate anaerobic bacteria—thioglycollate broth (TGB) with hemin and vitamin K
(3) For Mycobacterium spp.—Middlebrook 7H9 broth
(4) For Legionella spp.—Buffered charcoal yeast extract medium (BCYE)
(5) For fungi (e.g., Candida spp., Aspergillus spp.)—brain heart infusion (BHI) with chloramphenicol (8 μg/ml) and streptomycin (40 μg/mI)

### Example 4

### Kit for Determination of Antimicrobial Susceptibility of Microorganisms

Purpose

The purpose of this kit is to determine as quickly and as accurately as possible (for MOs identified by other means) the antimicrobial susceptibility of MOs by determination of the minimum inhibitory concentrations (MIC) of different antimicrobials.

This kit is analogous to Example 1: Kit for General Detection of Microorganisms in Blood Specimens. The general description and specific example presented in Example 1 apply, but with the following modifications:

(a) As a means of quality control, to show that the kit is working properly, ampoules with Mueller-Hinton broth (MHB) media are spiked with an optical density 0.5 McFarland units of bacterial suspensions using American Type Culture Collection (ATCC)-annotated MOs with known antimicrobial susceptibility and/or presence of resistance genes.

(b) For determination of antimicrobial susceptibility in blood specimens, chambers are filled with the requisite amount of MHB (2 ml in this example) also containing the following antimicrobial substances in ten serial dilutions 1:10, the highest concentration being ten times the estimated minimum inhibitory concentration inhibiting 90% of the MOs (MICgo). (In other versions of this kit, a smaller number of serial dilutions such as 3, 4, 5, 6, 7, 8, 9 covering, optionally, the same range as the series of 10 may suffice. A higher number of serial dilutions such as 15 or 20 are also within the scope of the present invention):
(1) Benzylpenicillin
(2) Oxacillin or nafcillin
(3) Ampicillin or amoxicillin
(4) Cefazolin
(5) Cefuroxim
(6) Ceftazidim
(7) Cefepim
(8) Imipenem
(9) Meropenem
(10) Clarithromycin
(11) Doxycyclin
(12) Clindamycin
(13) Tobramycin
(14) Amikacin
(15) Netilmicin
(16) Cotrimoxazol
(17) Nitrofurantoin
(18) Norfloxacin
(19) Ciprofloxacin
(20) Levofloxacin
(21) Moxifloxacin
(22) Vancomycin
(23) Teicoplanin
(24) Fusidinsafure
(25) Rifampicin
(26) Linezolid

Culture media may contain other additives including agents for maintaining the pH of the medium (salt and tris buffers), O₂-reducing agents (L-cystein), and specific nutrients (iron, factor X, factor V).

As in Example 1, 1 ml blood samples are placed in the ampoules containing MHB and the antimicrobial substances. Again, negative controls are also prepared, substituting 1 ml sterile PBS for the blood sample.

(c) The minimum inhibitory concentration (MIC) is defined as the lowest antimicrobial concentration which suppresses MO growth at a defined initial bacterial concentration (optical density of 0.5 McFarland), determined by observing the heat flow-time curve in serial antimicrobial dilutions 1:10.

The software determines in this example the minimum inhibitory concentration inhibiting 90% of the MOs (MIC₉₀) for each of the tested antimicrobial substances.

### Example 5

### Kit for Identification of Microorganisms

Purpose

The purpose of this kit is to identify microorganisms to the group, genus and/or species level in order to allow streamlining of antimicrobial therapy (i.e. switch from a broad-spectrum antibiotic to a targeted antimicrobial treatment) and to facilitate finding the origin of infection (primary focus or port of entry).

This kit is analogous to Example 1: Kit for General Detection of Microorganisms in Blood Specimens. The general description and specific example presented in Example 1 apply, but with the following modifications:

(a) The ampoules contain Mueller-Hinton broth (MHB) with additives to facilitate or inhibit growth of specific MOs. The additives for this kit include:
(1) Antimicrobial substances in concentrations higher than the minimum inhibitory concentration (MIC) for MOs of specific groups (e.g. glycopeptides suppress growth of the majority of Gram-positive MOs and aminoglycosides suppress growth of the majority of Gram-negative MOs, azoles suppress growth of the majority of fungi).
(2) Non-antimicrobial substances (e.g. hypertonic saline, extreme pH) to select specific MOs growing in their presence, but not others.
(3) Various types of growth media to compare the characteristics of heat flow-time curve in individual media, indicating the type of MO.

(b) The ampoules will be incubated at different growth conditions to identify types of MOs with specific requirements. These conditions can include:
(1) Different in-calorimeter incubation temperatures (e.g., 24° C., 37° C. and 40° C.)
(2) A slow, programmed change (e.g. 2° C./hour) in the incubation temperature (e.g. 24-40° C.) provided by a compatible calorimeter, since some MOs tend to enter the exponential growth phase at different temperatures.
(3) Changes in ampoule head space initial atmosphere (e.g., ambient air, reduced oxygen tension, 5-10% carbon dioxide)

(c) With the equipment used in this example, it is not possible to agitate the contents of the sealed ampoules. However, compatible calorimeters may be used which allow agitation of the culture within the ampoule. Comparison of results for ampoules which are non-agitated vs. agitated may also aid in identification since in some cases the effects of agitation are different for different MOs.

The approach taken in this kit is to follow logic schemes to broadly identify MOs on the basis of whether growth is encouraged or inhibited by certain combinations of environmental variables. In this embodiment advantage can be taken of calorimetry allows current (“real-time”) growth data to be continuously available. The kit also allows for real-time quantitative analysis of the changes in growth rates.

The software helps in this example the kit user to decide which MO is present in the ampoules on the basis of combinatorial analysis of the additives and conditions facilitating or inhibiting growth.

Once given the above disclosure, many other features, modifications, and improvements will become apparent to the skilled artisan. Such other features, modifications, and improvements are therefore considered to be part of this invention, the scope of which is to be determined by the following claims:

1. Method for assessing microbial growth in a sample comprising
(a) providing at least one medium selectively promoting or suppressing growth of at least one microorganism or at least one type of microorganisms,
(b) combining an aliquot of said sample with said at least one medium to produce a sample mixture,
(c) collecting heat flow signals from said sample mixture over time to produce a heat flow signal curve,
(d) determining from said heat flow signal curve whether an indicator function indicative of growth, growth rate, identity or quantity of said at least one microorganism or at least one type of microorganism exists, and if so,
(e) processing said indicator function to determine growth, growth rate identity and/or quantity of said of at least one microorganism or at least one type of microorganism.

2. A kit for assessing microbial growth in a sample comprising:
(a) at least one medium selectively promoting or suppressing growth of at least one microorganism or at least one type of microorganisms,
(b) at least one receptacle for combining said at least one medium with an aliquot of said sample to produce a sample mixture,
(c) instructions to collect heat flow signals from said sample mixture over time to produce a heat flow signal curve,
(d) a software component comprising
(i) a determining function that determines whether said heat flow signal curve displays an indicator function indicative of growth, growth rate, identity or quantity of said at least one microorganism or at least one type of microorganism, and
(ii) a processing function that processes any indicator function of (i) to determine growth, growth rate, identity and/or quantity of said at least one microorganism or at least one type of microorganism.

3. A system for assessing microbial growth in a sample comprising:
(a) at least one medium selectively promoting or suppressing growth of at least one microorganism or at least one type of microorganisms,
(b) at least one receptacle for combining said at least one media with an aliquot of said sample to produce a sample mixture,
(c) a calorimeter that collect heat flow signals from said sample mixture over time to produce a heat flow signal curve,
(d) a software component comprising
(i) a determining function that determines whether said heat flow signal curve displays an indicator function indicative of growth, growth rate, identity or quantity of said at least one microorganism or at least one type of microorganism, and
(ii) a processing function that processes any indicator function of (i) to determine growth, growth rate, identity and/or quantity of said at least one microorganism or at least one type of microorganism.

4. The method of claim 1, wherein at least 2, preferably at least 6, more preferably at least 12 media are provided.

5. A kit according to claim 2, wherein at least 2, preferably at least 6, more preferably at least 12 receptacles and media are part of the kit and each of said receptacles is, optionally preloaded with one of said media.

6. The method according to claim 1, wherein the indicator function is determined in real-time and is, optionally, processed in real-time.

7. The method according to claim 1, wherein in (b) a control is provided to produce a heat flow signal curve of said control and wherein said indicator function of (d) is a significant difference between values of the heat flow signal curve of the sample mixture and values of the heat flow signal curve of the control.

8. The method according to claim 7, wherein a significant difference constitutes a difference that is about five times, six times, seven times, eight times, nine times, ten times or more the detection limit of an instrument measuring said heat flow signals.

9. The method according to claim 1, wherein said indicator function is a lag until growth starts, an rise or fall of a growth curve, a peak in the growth curve, the overall shape of one or more curves or parts thereof or a combination thereof.

10. The method of claim 9, wherein the presence of microbial growth in said sample is determined by processing the rise or fall of the growth curve, logarithmic data is computed from said rising or declining part of said heat flow signal curve and a linear regression is performed on said logarithmic data to calculate a R² value to determine a degree of linearity of said logarithmic data.

11. The method of claim 10, wherein a R² value of about 0.9 and above, preferably about 0.95 and above or about 0.99 and above indicates and/or confirms microbial growth.

12. The method of claim 9, wherein the lag is the indicator function for initial concentration of microorganisms in said sample, which is processed to correlate the time of the lag to the initial concentration of microorganisms.

13. The method of claim 9, wherein the overall shape of the curve is the indicator function of quantity in said sample which is processed to calculate the area under the curve and thus determine the quantity of microorganisms in the sample.

14. The method according to claim 1, wherein precollected heat flow signal data from different microorganisms is provided to ascertain the growth, growth rate, identity or quantity of said microorganism.

15. The method according to claim 1 further comprising collecting heat flow signals from a control and subtracting the so obtained values from corresponding values of the heat flow signal curve of said sample mixture to accelerate detection and determination of an indicator function and/or to increase overall accuracy.

16. The method according to claim 1, wherein a time interval between producing said sample mixture and determining growth, growth rate, identity and/or quantity of said microorganism are smaller than about 10 hours, about 9 hours, about 8 hours, about 7 hours, about 6 hours, about 5 hours, about 4 hours, about 3 hours, about 2 hours, about 1 hour, about 45 minutes, about 30 minutes or about 15 minutes.

17. The system according to claim 3, wherein said heat flow signal curve is based on between about 5 and about 250, between about 5 and about 100, between about 5 and about 50 point measurements of heat flow signals.

18. The system according to claim 3, wherein said heat flow signals are detected by a calorimeter having chambers, wherein the calorimeter is stabilized at a specific temperature or over a continous range of temperatures and the chambers are calibrated to produce accurate measurements of said heat flow signal at said specific temperature.

19. The method according to claim 1, wherein a time interval from producing the sample mixture and the insertion of the sample mixture into a calorimeter chamber is kept approximately constant between a first sample mixture and any subsequent sample mixtures comprising aliquots of the same sample.

20. The method according to claim 3, wherein said sample mixture is placed into a calorimeter chamber at an equilibration position for a predetermined equilibration time to reach a predetermined temperature.

21. The system according to claim 20, wherein, following elapse of the equilibration time, the sample mixture is lowered to a measurement position for a predetermined stabilization time until extraneous heat flow signals subside.

22. The system of claim 21, wherein control heat flow signal values collected for one or more controls is subtracted from the corresponding values of the heat flow signal curve of the sample mixture to reduce said stabilization time and wherein, when heat flow signals of more than one control are collected, the mean of those control heat flow signal values is subtracted.

23. The kit according to claim 2, wherein the sample is cerebrospinal fluid.

24. The kit of claim 23, wherein the at least one medium is brain heart infusion, Barbour-Stoenner-Kelly II medium, Middlebrook 7H9 broth, Brucella broth, optionally with sodium bisulfite or University of Vermont modified Listeria enrichment broth.

25. The method of claim 2, wherein the sample is cerebrospinal fluid and, wherein growth of meningitis bacteria Streptococcus pneumoniae and/or Neisseria spp. in said sample mixture is assessed and wherein a peak value of the respective heat flow signal curves allows for differentiation between the two bacteria.

26. The kit according to claim 2, wherein a series of media selectively suppressing growth are provided and are combined with aliquots of one sample, and wherein said indicator function is the change of growth in one of said media relative to the others in the series, which is processed to determine susceptibility to and/or minimum inhibitory concentration of a suppressor agent in said medium.

27. The kit according to claim 26, wherein said series of media comprises serial dilutions of at least one suppressor agent so that a minimum inhibitory concentration of the suppressor agent in said medium can be determined.

28. The kit according to claim 2, wherein a series of media selectively suppressing or promoting growth are provided and are combined with aliquots of one sample, and wherein said indicator function is the absence or presence of growth in one of said media relative to the others in the series, which is processed to determine the identity of a microorganism in said sample.

29. The method of claim 1, wherein a series of media are combined with aliquots of one sample and wherein said indicator function is a combination of the obtained heat curves which is processed to determine the identity of a microorganism in said sample.

30. (canceled)

31. The kit according to claim 2, wherein in (b) a control is provided to produce a heat flow signal curve of said control and wherein said indicator function of (d) is a significant difference between values of the heat flow signal curve of the sample mixture and values of the heat flow signal curve of the control.
